(19) United States
(12) Patent Application Publication
(10) Pub. No.: US 2024/0252605 A1
(43) Pub. Date: Aug. 1, 2024

(54) TICK MOUTHPART ANTIGENS AS EFFECTIVE ANTI-TICK VACCINES

(71) Applicant: University of Maryland, College Park, College Park, MD (US)

(72) Inventor: Utpal PAP, Ellicott City, MD (US)

(21) Appl. No.: 18/560,594

(22) PCT Filed: May 16, 2022
(86) PCT No.: PCT/US2022/029441

§ 371 Date: Nov. 13, 2023

Related U.S. Application Data
(60) Provisional application No. 63/201,846, filed on May 14, 2021.

(51) Int. Cl. A61K 39/00; A61P 33/14; C07K 14/435

(52) U.S. Cl. CPC A61K 39/0003; A61P 33/14; C07K 14/43527

(57) ABSTRACT

Provided are compositions and methods used for combating ticks and transmission of tick-borne pathogens to tick hosts. The compositions include tick mouthpart protein antigens. The method of using the compositions results in decreased tick persistence time, decreased tick feeding, and decreased tick-borne pathogen transmission from tick to the host.

### CROSS-REFERENCE TO RELATED APPLICATION

This application claims priority to U.S. provisional patent application No. 63/201,846, filed May 14, 2021, the entire disclosure of which is hereby incorporated by reference.

### SEQUENCE LISTING

The instant application contains a Sequence Listing which has been submitted electronically in ASCII format and is hereby incorporated by reference in its entirety. Said ASCII file, created on May 16, 2022, is named UMD_TMPr1_PCT_ST25.txt, and is 66,655 bytes in size.

### FIELD

The presently disclosed subject matter relates generally to preventing or reducing tick biting and the transfer of infectious agents from ticks to their hosts.

### BACKGROUND

The blacklegged tick (Ixodes scapularis) is one of the major vector species that is highly prevalent in many parts of North America. In fact, I. scapularis and related tick species are distributed all across Europe and parts of Asia and surpass most other arthropod vectors in the transmission of a number of serious human diseases—including viral, bacterial, and parasitic pathogens—in the United States and many other parts of the globe¹. The most common Ixodes tick-transmitted infection is Lyme disease or Lyme borreliosis, which is caused by a unique group of spirochete bacteria called Borrelia burgdorferi sensu lato. There are more than 450,000 new cases estimated to occur every year in the U.S. alone², where the medical cost associated with the management of Lyme disease and its sequelae approaches $1.3 billion every year^{2a}. The infection has now been reported in more than 80 countries and is considered to be one of the most common vector-borne diseases in the Northern Hemisphere. Besides Lyme disease, there are additional Ixodes tick-borne infections that are also frequently reported in the United States and in Europe, such as anaplasmosis, babesiosis, tick-borne encephalitis, and Powassan virus-related disease^{3,4}. In addition to existing infections, new pathogens have been recently discovered that are also transmitted by Ixodes tick species, such as B. mayonii⁵ and B. miyamotoi⁶, which can cause Lyme disease or similar human infections. During a blood meal engorgement, ticks can transmit pathogens into the skin of their mammalian hosts, including humans and domesticated animals. After a tick punctures the skin and secretes saliva and possibly other secretions from mouthparts, any pathogens it may carry are also deposited in the host dermis, which can then disseminate to a variety of distant organs within the host, where they can elicit inflammatory responses.⁷⁻¹⁰.

Ticks are ancient and unique disease vectors. They can parasitize almost any vertebrate class across the globe and are considered to be second only to mosquitoes as vectors of serious human infectious diseases. The I. scapularis complex transmits a wide array of viral, bacterial and eukaryotic pathogens to humans. About 95 percent of reported cases of vector-borne disease are associated with ticks, making these the most medically important group of arthropods in the United States^{10a}. What distinguishes the tick among other medically important arthropods is the duration of its feeding on vertebrate hosts, which ranges from several days (larval and nymphal stages) to over a week (adult stage), along with its ability to harbor and transmit multiple pathogens (referred to as co-infection), as well as its ability to maintain a diverse microbiome in the gut, including infectious agents¹¹. Tick feeding is a slow yet extensive process, and the transmission of pathogens occurs during these complex episodes of blood meal engorgement. Many tick-borne pathogens that colonize the tick salivary gland can transmit to the host within hours of tick attachment, whereas other pathogens transmit much more slowly, such as B. burgdorferi, which colonizes the tick gut and transmits 48 hours after the onset of the tick feeding process¹².

When a questing tick finds a host and initiates its blood meal engorgement, it first inserts its mouthparts¹³ into the host dermis and secretes a series of pharmacologically active substances that are produced by the tick's salivary glands. These molecules could also be produced from tissues or cells associated with the tick hypostome and chelicerae, which are inserted in the host dermis and are potentially in direct contact with the host blood vasculature. Once transferred into the host, these tick molecules expedite pathogen transmission via modulation of the host's physiological and immunological responses. The tick mouthpart components, particularly the hypostome and chelicerae, including its secreted molecules, are therefore integral to tick feeding, as they allow the tick to firmly anchor into the host's skin and successfully acquire its blood meal. After feeding, the bodyweight of a fully engorged tick increases from its unfed stage by a factor of 30-100-fold. Together, the functions of the tick mouthparts, including the constituent cells and secreted products, are critical not only to tick physiology and blood meal engorgement, but also for optimal survival of the microbes and pathogens in the gut. Further, a single tick can carry multiple pathogens (for example, B. burgdorferi and Anaplasma phagocytophilium), thus, after a blood meal engorgement can elicit co-infection (such as infections that cause Lyme disease and anaplasmosis) in susceptible mammalian hosts, including humans. Despite substantial efforts over the past several decades, human vaccines against most tick-borne illnesses remain unavailable. Thus, there is an ongoing and unmet need for improved compositions and methods for inhibiting tick biting and associated infections in humans. The present disclosure is pertinent to this need.

### BRIEF SUMMARY

The present disclosure provides a new approach to combating ticks. Unlike previous tick vaccines, which rely on antigens expressed by pathogenic microbes that are present in ticks, or tick antigens from the gut and saliva protein, the present disclosure demonstrates that proteins and segments thereof from tick mouthparts can be used to stimulate an anti-tick response in tick hosts and provides a segment of a tick mouth protein that has superior anti-tick function relative to other tick mouth proteins. In this regard, in one aspect, the disclosure provides a method for inhibiting the persistence of tick attachment to a mammalian host, and/or inhibiting transmission of one or more tick-borne pathogens from the tick to the mammalian host. The method comprises administering to the mammalian host an isolated or recombinantly produced protein that has at least 95% sequence identity with the sequence of SEQ ID NO: 1, said SEQ ID NO: 1 referred to herein as TMP-r1. Use of vaccine formulations comprising TMP-r1 is demonstrated to reduce the time the tick remains attached to the mammalian host, reduce the amount of blood consumed by the tick, and furthermore inhibit transmission tick-borne pathogens to the mammalian host, such as Borrelia burgdorferi. The compositions and methods are suitable for use with any animal that is susceptible to tick bites, including but not necessarily limited to humans, canines, felines, and equine animals. Avian animals may also benefit from the described approaches. Vaccine compositions, which comprise at least one of the described tick mouth proteins are also provided. Also included in the disclosure are isolated and recombinantly produced proteins, fusion proteins, expression vectors encoding the proteins, and a method of making the described proteins. The method of making the described proteins comprises expressing the protein from an expression vector in a plurality of prokaryotic cells and separating the expressed protein from the cells.

### BRIEF DESCRIPTION OF THE FIGURES

FIG. 1A. Diagram showing an image of the tick-bite site of the mouse dermis (inset shows the whole tick for orientation) while the bottom panel represents a highly magnified view of the tick mouthparts.

FIG. 1B. The diagram shows an experimental strategy to identify anti-tick vaccines using molecular components of tick mouthparts as the target.

FIG. 1C. List of abundant proteins identified in the mouthparts that include a class of six proteins of unknown functions.

FIG. 2. Identified Tick mouthpart associated protein (TMP)s. The amino acid sequences shown in the amino acid alignments in FIG. 2, from top to bottom, are SEQ ID NO:s 3-8, respectively. A multiple sequence alignment showing identification of known domains in TMP proteins. The vitellogenin-like domain (VG), and a domain of unknown function (DUF) are indicated as labeled and boxed. TMP-r1 is amino acids 216-455 in the top sequence in the alignment (GI 215504085); TMPr-2 is amino acids 670-950 in the second sequence from the top in the alignment (GI 215504084); TMPr-3 is amino acids 1424-1532 in the second sequence of the alignment (GI 215504084).

FIG. 3. The DNA and Protein sequence for TMP-r1 used as a vaccine. The DNA sequence is SEQ ID NO:2. The amino acid sequence is SEQ ID NO:1.

FIGS. 4A and 4B. Expression of Tick Mouth Protein in tick tissues and during blood meal engorgement. Nymphal Ixodes ticks were collected from rodents during blood meal engorgement at various time points of feeding (24-96 hours) and dissected to isolate various tissues. TMP expression was assessed using primers specific to TMP-r1 via real-time PCR analysis and normalized against tick actin gene copies. FIG. 4A and FIG. 4B show relative levels of TMP-r1 specific transcripts at various tick tissues and feeding time points, respectively.

FIGS. 5A and 5B. Recombinant TMPr-1 and antibodies. FIG. 5A. Production of recombinant TMP-r1 in the E. coli system. Purified recombinant TMP-r1 (arrow) was resolved in the SDS-PAGE gel, transferred onto nitrocellulose membrane, stained with Ponceau S (left lane), and subsequently immunoblotted with anti-TMPr1 antibodies generated in mice immunized with TMP-r1. FIG. 5B. Immunization of TMPr1 induces high-titer antibodies. Antibody titers for TMP-r1 serum samples from individual TMPr1-immunized guinea pigs were serially diluted from 1:1000 to 1:1,000,000 and probed against recombinant TMPr1 coated ELISA wells.

FIG. 6. Data showing immunization of mice with individual regions of tick mouthparts antigens results in higher tick detachment. Mice were immunized with 10 uG of each tick antigens in PBS (one primary and two booster immunizations) and then exposed to ticks (24 ticks per group in the first experiment and 15-30 ticks per group in the second experiment). Mice immunized with adjuvant only (PBS) were used as controls. The data represents the number (percentage) of ticks recovered from each group. Compared to control where 71-83% of ticks were recovered as fed nymphs, the ticks parasitizing antigen immunized groups are less successfully fed and recovered as 38-77% engorged ticks.

FIG. 7. Tmp-r1 immunization prevents successful tick engorgement in guinea pigs. The animals were immunized with TMP-r1 protein or control (either adjuvant only, or another tick protein), after three successive immunizations and then challenge with ticks. Note a significant detachment of ticks was observed in animals that were immunized with TMP-r1, compared to the control group.

FIG. 8. Tmp-r1 immunization is enough to generate protective immunity against successful tick engorgement in guinea pigs. The animals were immunized with TMP-r1 protein, a cocktail of three TMP proteins (TMP-r1+TMP-r2+TMP-r3) or control (adjuvant only), or additional control (another tick gut protein)′ after three successive immunizations, the animals were challenged with ticks and collected as repleted ticks. TMP-r-1 vaccination produced the most effective protective immunity against tick bites.

FIG. 9. Tmp-r1 immunization prevents successful tick engorgement in guinea pigs. As detailed in FIG. 7, the animals were immunized with TMP-r1 protein or control (either adjuvant only, or another tick protein), after three successive immunizations and then challenged with ticks. The data shown are the weight of engorged ticks. Some of the ticks placed on TMP-r1 immunized animals were unable to feed.

FIGS. 10A and 10B. TMP-r1 immunization impairs B. burgdorferi transmission from ticks to rodent hosts. The guinea pigs were immunized with TMP-r1 or control, as detailed in previous figures and challenged with B. burgdorferi-infected nymphal Ixodes scapularis ticks (5-10 ticks/animals). After 4 weeks, the animals were assessed for B. burgdorferi infection by RT-qPCR analysis of skin samples (FIG. 10A) or by immunoblot analysis of spirochete lysate and serum samples collected from immunized and infected animals (FIG. 10B). The immunoblot from positive controls (serum from infected animals) and negative controls (naïve serum and antibody controls) are shown. Note, immunoblot patterns of individual animals immunized with TMPr1 is more comparable to that of naïve immunoblot, as compared to ones immunized with Control proteins, suggesting less infection. The data show that TMP-r1 vaccination significantly protects immunized hosts from B. burgdorferi infection.

### DETAILED DESCRIPTION

Although claimed subject matter will be described in terms of certain embodiments/examples/aspects, other embodiments/examples/aspects, including embodiments/examples/aspects that do not provide all of the benefits and features set forth herein, are also within the scope of this disclosure. Various structural, logical, and process step changes may be made without departing from the scope of the disclosure.

Every numerical range given throughout this specification includes its upper and lower values, as well as every narrower numerical range that falls within it, as if such narrower numerical ranges were all expressly written herein. All ranges provided herein include all values that fall within the ranges to the tenth decimal place, unless indicated otherwise. The sequence described under any reference to an amino acid or nucleotide sequence by way of a database entry is incorporated herein by reference as the sequence exists in the database as of the effective filing date of this application or patent.

Throughout this disclosure, the singular form encompasses the plural and vice versa.

The disclosure includes amino acid sequences that are from 80%-99% similar to the described amino acid sequences, and includes amino acid sequences that include insertions and deletions, and conservative amino acid substitutions, provided the protein that comprises differences between the described sequences retains its described effects. All polynucleotides encoding the described proteins are included in this disclosure. The disclosure includes expression vectors encoding the described proteins, and cells that are modified to include an expression vector such that the described proteins can be produced and separated from the cells.

Any amino acid sequence described herein can comprise or consist of the described sequence. Any composition that comprises a described protein may be provided such that the described protein is the only protein that comprises a tick antigen in the composition.

The present disclosure provides a previously unreported and new concept of utilizing molecular components of tick mouthparts as components of anti-tick vaccines.

The disclosure relates in part to an analysis of the most abundant and potentially immunogenic components of the tick mouthparts, such as proteins. The disclosure demonstrates among other embodiments, immunization of hosts with selected proteins and protein segments to assess the ability of the proteins to, among other aspects, combat tick biting and the concomitant development of infections in the tick host by tick-borne pathogens.

As discussed above, the present disclosure represents a new approach to combating tick infections, namely, by providing and using one or more proteins that are from tick mouthparts as an agent(s) to deter tick attachment, inhibit attachment time, and inhibit transmission of tick-borne pathogens to a host, among other functions that are described herein. The disclosure provides an analysis of several tick mouthpart proteins, such proteins are referred to herein collectively as a Tick mouthpart associated proteins (TMPs). The disclosure unexpectedly reveals that a segment of one of the identified TMPs, referred to herein as TMP-r1, has superior anti-tick properties relative to the other identified TMPs.

The amino acid sequence of TMPr-1 is:

(SEQ ID NO: 1)
TSQVKYRLDGTPEHYVINHACATSENVFRPFGQGKTFVAQLNRTLDLEE
VHDANTDTQLPEDLEKVHHIAQTFPESDEVESLEELKHVNRYVTTFDLS
TDKDKFISGLNHLAALEYEDSDIKDVHSKESGGLNFLILFGSLASMPFE
DIAHVYEQAVANAPEASKSQVRKVFLDLLSAVGNNPHAAFGLQLVKEDK
LTDEEAEHFLAKLALNLKENSPALLTELAEVCEHVKPKRPVWVN.

A representative DNA sequence encoding SEQ ID NO:1 is:

(SEQ ID NO: 2)
ACCTCTCAGGTCAAGTACAGACTCGACGGAACTCCAGAACACTACGTCA
TCAACCACGCCTGCGCCACCTCAGAGAATGTCTTCAGGCCCTTCGGTCA
AGGGAAAACCTTCGTTGCCCAGCTCAACCGTACCTTGGATTTGGAGGAG
GTGCACGACGCCAACACCGACACTCAGCTTCCGGAGGACCTGGAGAAGG
TGCACCACATCGCGCAGACATTCCCCGAGTCTGATGAGGTGGAAAGCCT
GGAGGAACTGAAGCACGTCAACCGCTACGTGACCACCTTTGATCTCTCG
ACCGACAAAGACAAGTTCATCTCTGGTCTCAACCACCTTGCTGCATTGG
AGTACGAAGATTCCGACATCAAGGACGTTCACAGCAAGGAAAGCGGTGG
ACTAAACTTCCTGATTTTGTTCGGCTCGCTTGCCTCCATGCCCTTTGAA
GATATCGCCCACGTGTACGAGCAAGCCGTTGCCAATGCACCGGAAGCAA
GCAAGAGTCAAGTCAGAAAGGTGTTCCTGGACCTGCTCTCAGCTGTCGG
AAACAACCCTCACGCAGCGTTCGGTCTGCAACTGGTCAAGGAAGACAAA
CTCACTGACGAAGAGGCTGAGCACTTCCTCGCCAAGCTGGCGCTGAACC
TTAAGGAGAACAGCCCCGCTCTTCTGACCGAACTTGCCGAGGTGTGTGA
GCACGTGAAGCCTAAGCGACCAGTATGGGTCAAC.

The disclosure includes isolated and recombinantly produced TMPs. In embodiments, the described TMPr-1 protein may be provided as a pharmaceutical formulation. A pharmaceutical formulation can be prepared by mixing the described protein with any suitable pharmaceutical additive, adjuvant, buffer, and the like. Examples of pharmaceutically acceptable carriers, excipients, and stabilizers can be found, for example, in Remington: The Science and Practice of Pharmacy (2005) 21st Edition, Philadelphia, PA. Lippincott Williams & Wilkins, the disclosure of which is incorporated herein by reference. In embodiments, the pharmaceutical formulation comprises a vaccine. In an embodiment, the vaccine comprises a suitable adjuvant. In an embodiment, the vaccine is provided as a composition that comprises the TMPr-1 protein and an immuno-effective amount of an adjuvant. In an embodiment, the adjuvant comprises Freund's adjuvant or Freund's incomplete adjuvant. In embodiments, the adjuvant comprises an alum, or one or a combination of aluminum salt adjuvants, a non-limiting example of aluminum salt adjuvants being aluminum phosphate.

In certain approaches, a described protein is modified for prophylactic or therapeutic approaches. In embodiments, the protein is stapled, cyclicized, or multimerized. In embodiments, the described TMPr-1 protein is provided as a component of a fusion protein. The fusion protein may comprise, in addition to the TMPr-1 amino acids, additional amino acids that improve the efficacy of the protein. In embodiments, the additional amino acids extend the half-life of the fusion protein. In embodiments, the fusion protein comprises one or more bacterial or other tick antigens. In one embodiment, the TMPr-1 protein is fused to a bacterial ferritin to, for example, create protein-ferritin nanoparticles. In embodiments, the TMPr-1 protein is fused to a different TMP described herein. In embodiments, a described protein is modified to include additional amino acids used for purification, including any suitable affinity tag, representative examples of which include histidine tags, such as at least four histidines, such as 4, 5, 6, 7, 8, 9, or 10 histidines, biotinylation, glutathione-S-transferase, a FLAG-tag, an epitope tag, and the like.

In addition to fusion proteins, the disclosure includes combining the described TMPr-1 protein with other tick and/or bacterial antigens to create multi-valent vaccines. In embodiments, the composition comprises a bacterial protein, such as a lipoprotein produced by a pathogenic bacteria, or a derivative of such a protein. In embodiments, the composition thus may further comprise bacterial OspA, OspB, OspC, a combination thereof, or a TMPr-1 comprising all or antigenic segments of any of the OsP proteins. The described proteins may be produced by or derived from proteins produced by, for example, B. burgdorferi. In embodiments, a composition of this disclosure comprises the described TMPr-1 and live mutant attenuated pathogenic bacteria that are modified versions of bacteria that are transferred by ticks to the subjects they bite. In embodiments, a combination of TMPs that includes TMPr-1 is used.

In embodiments, an effective amount of a TMPr-1 protein described herein is administered to an individual. In embodiments, an effective amount can be determined based on the present disclosure by those skilled in the art, taking into account certain factors such as the type of individual being vaccinated, as well as any of the size, gender, and/or age of the individual. In embodiments, the effective amount is an amount that is sufficient to achieve the described effect in single or multiple doses. In embodiments, an effective amount means an amount sufficient to prevent, or reduce by at least about 30 percent, or by at least 50 percent, or by at least 90 percent, any sign or symptom of tick attachment, and/or any sign or symptom of pathogen infection that is associated with tick bites. In embodiments, the number of ticks attached to an individual who has received an effective amount of the vaccine is reduced, relative to the number of ticks attached to an individual who has not received the vaccine. In embodiments, one or more ticks detach from the vaccinated individual in a shorter period of time relative to the attachment time for an unvaccinated individual. In embodiments, an effective amount results in a tick consuming less blood, relative to the amount of blood consumed by a tick attached to an unvaccinated individual. In embodiments, an effective amount results in inhibition or prevention of infection by a pathogen carried by the tick, representative examples of such pathogens include but are not necessarily limited to pathogenic spirochete bacteria. In embodiments, infection by Borrelia burgdorferi including any serotype thereof is inhibited or prevented. In embodiments, infection by B. burgdorferi sensu lato is inhibited or prevented. In embodiments, infection by B. mayonii, or B. miyamotoi, or Anaplasma phagocytophilium is inhibited or prevented. In embodiments, administering a described vaccine composition inhibits the development or reduces the severity of any of Lyme disease, anaplasmosis, babesiosis, tick-borne encephalitis, or Powassan virus-related disease. In embodiments, administration of a composition comprising the described protein elicits antibodies, which are neutralizing against a tick-borne pathogen.

The type of tick against which the described TMPr-1 protein and compositions comprising the protein is effective is not particularly limited. In embodiments, the type of tick is any species of Ixodes. In one embodiment, the tick is a blacklegged tick (Ixodes scapularis). In an embodiment, the tick is Amblyomma americanum.

The described TMPr-1 protein and compositions comprising it or other components as described herein can be administered to an individual using any suitable route, including but not limited to subcutaneous administration, oral administration, or intravenous administration. In an embodiment, the disclosure provides a microneedle array coated with a described protein for use in subcutaneous administration. In embodiments, administration of a described vaccine does not induce inflammation in the vaccinated individual, such as inflammation of the skin of the individual.

In certain embodiments, a described protein is introduced to an individual using a polynucleotide that encodes the protein. In a non-limiting embodiment, the polynucleotide is an mRNA that may be delivered with a suitable delivery reagent, including but not necessarily limited to liposomal nanoparticles.

The individual to which a described protein or composition comprising a described protein is administered is not particularly limited. In embodiments, the composition is administered to a mammal or an avian animal. In embodiments, the composition is administered to a human. In alternative embodiments, a composition of this disclosure is administered to a canine, a feline, an equine animal, a rodent, an avian animal, or bovine animals, such as cattle, including but not limited to dairy cattle.

The following Examples are intended to illustrate but not limit the disclosure.

### Example 1

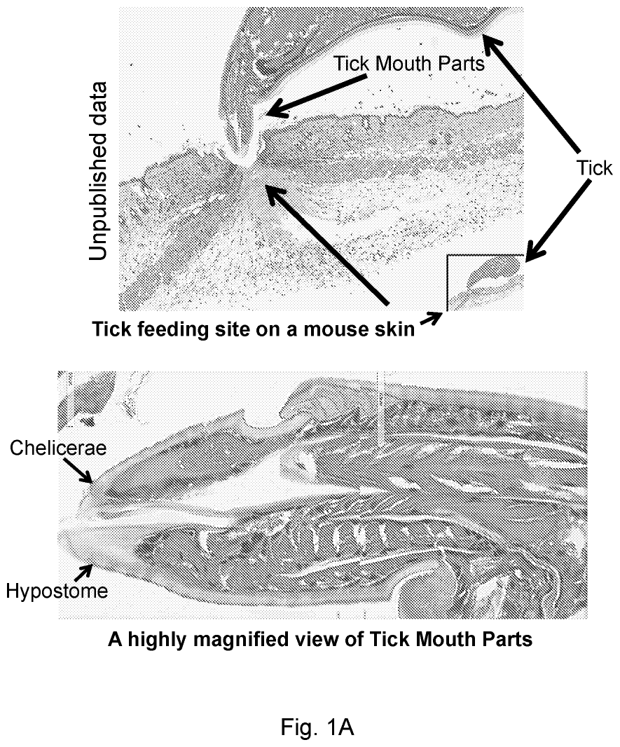

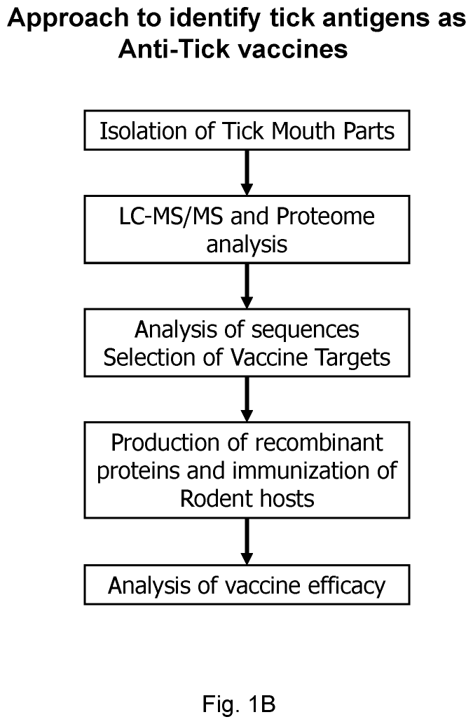

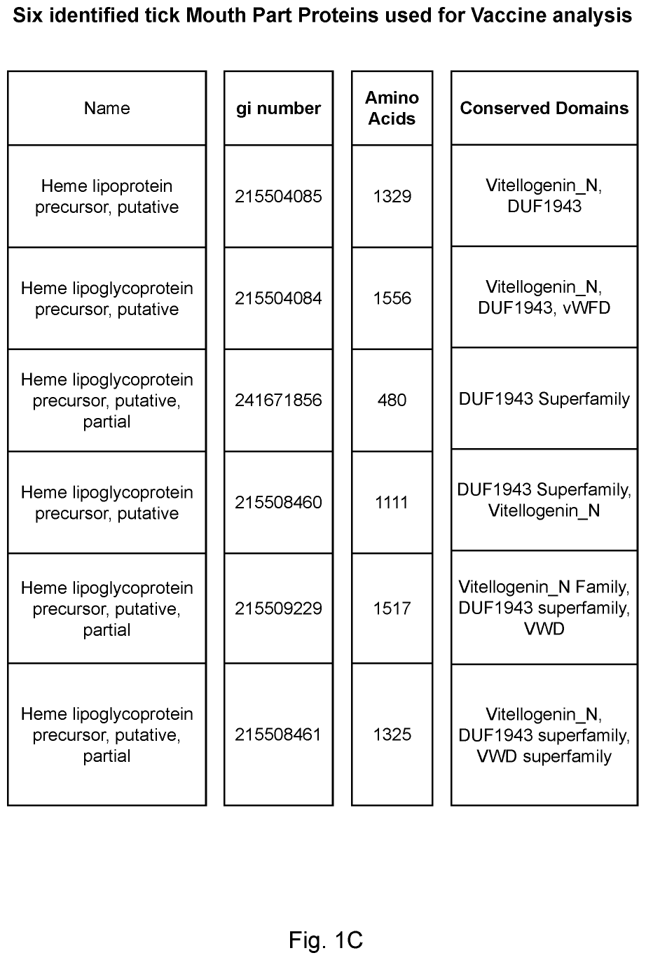

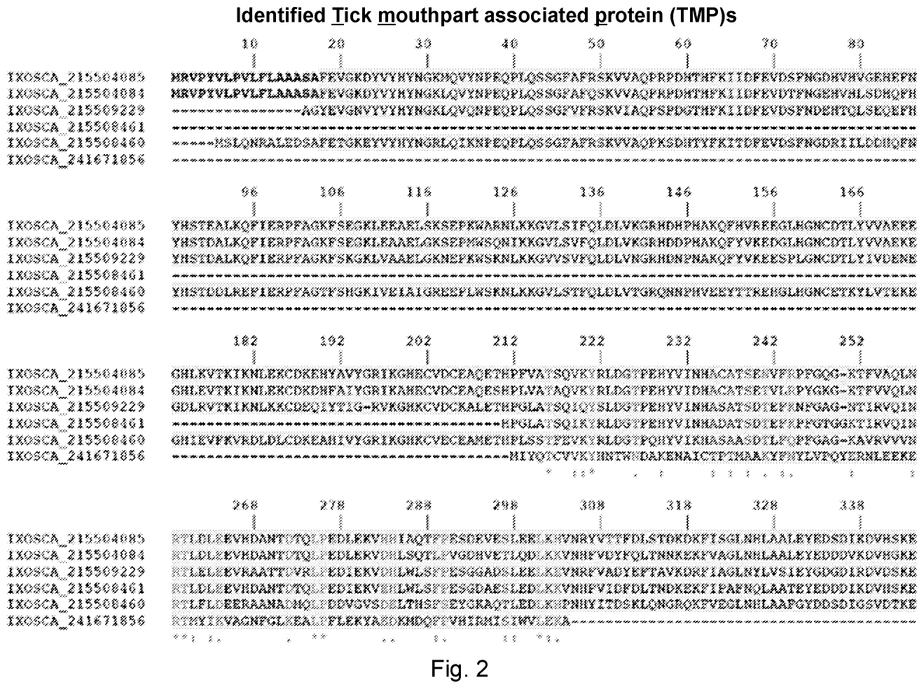

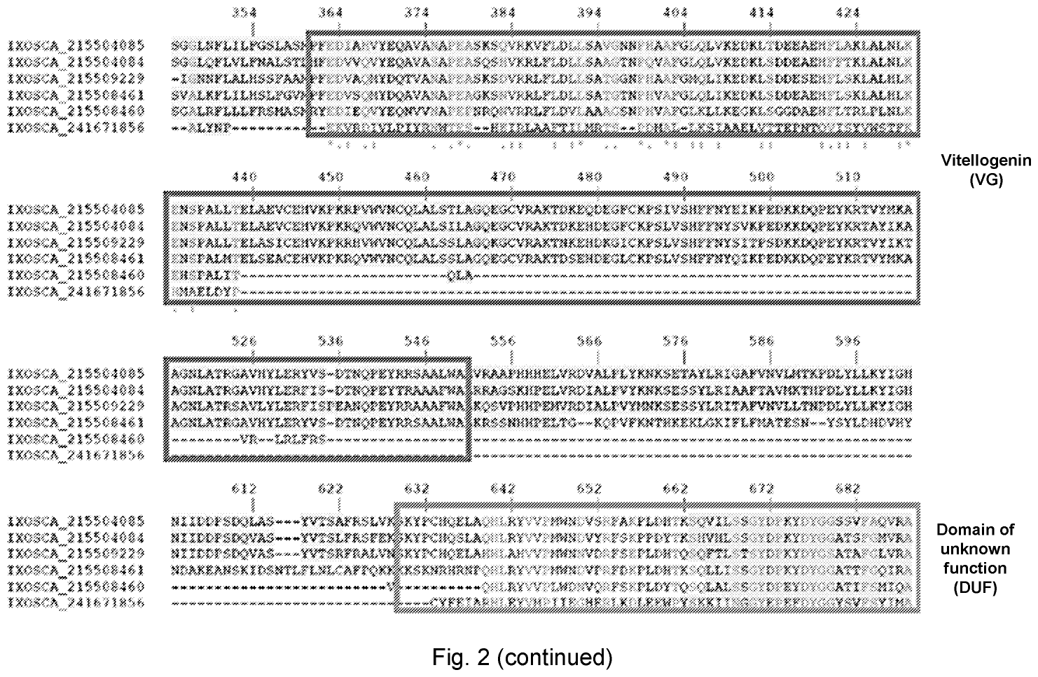

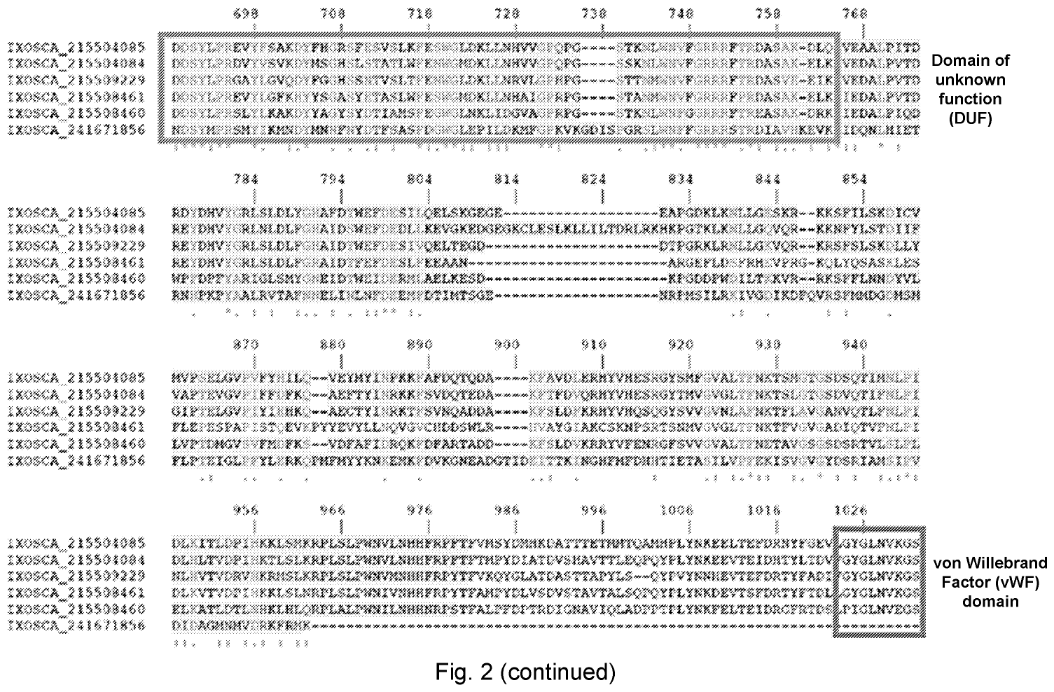

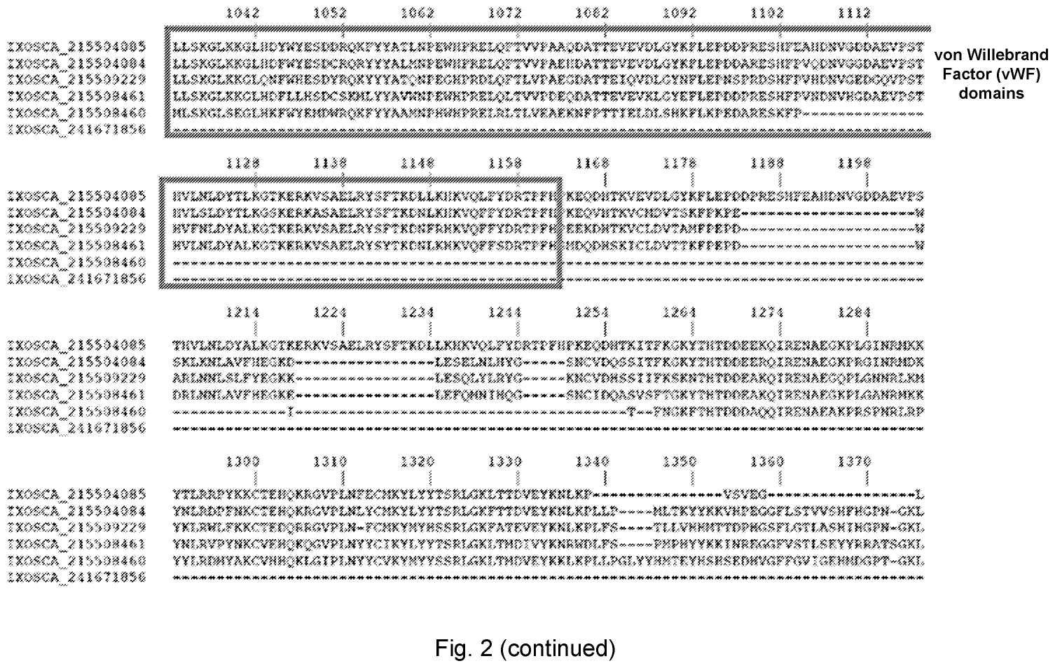

To assess anti-tick vaccine candidates, we allowed I. scapularis nymphs to engorge on naïve mice. About 24 hours after tick placement, when the nymphs had successfully inserted their mouthparts into the mouse dermis and initiated the feeding process we removed the partially-fed ticks from the host. Under a high power zoom stereo dissecting microscope, we separated and isolated the mouthpart structures from these partially-fed ticks (FIG. 1A) and then processed them for proteome identification using a nano LC-MS/MS developed by our laboratory¹⁵. The approach is shown in FIGS. 1A and 1B. We discovered that some of the most abundant proteins identified in the mouthparts are a class of six proteins of unknown functions (FIG. 1C), termed herein as tick mouthpart associated proteins (TMPs) (FIG. 2). Further bioinformatics revealed that the identified TMPs harbor one to three domains and conserved regions that are common in most proteins, such as the Vitellogenin (VG) domain, domain of unknown function (DUF), and von Willebrand Factor (VWF) domains (Box 1 and FIG. 2). Using additional bioinformatic analysis, we selected three vaccine candidates, referred to herein as TMP-r1, TMP-r2 and TMP-r3, which overlap with the VG, DUF and VWF domains, respectively. As further described below, amongst all candidate proteins we tested by, only TMP-r1 exhibited properties that are suitable for use as a vaccine. as immunization with it prevented successful tick blood meal engorgement. Thus, the disclosure reveals a specific region of TMP, designated as TMP region 1 (TMP-r1), a ˜24 kDa protein antigen, and elicits the most effective anti-tick vaccine response of the proteins tested.

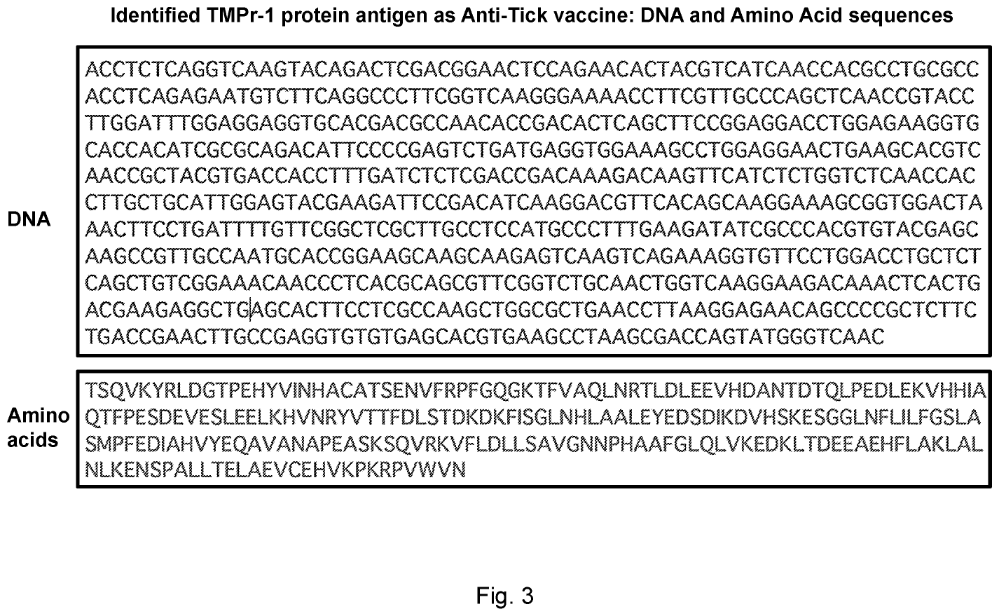

The nucleotide and amino acid sequence of TMP-r1 is shown in FIG. 3.

The sequence of a segment of the TMP protein referred to as TMPr-1 in FIG. 3 is:

(SEQ ID NO: 1)
TSQVKYRLDGTPEHYVINHACATSENVFRPFGQGKTFVAQLNRTLDLEE
VHDANTDTQLPEDLEKVHHIAQTFPESDEVESLEELKHVNRYVTTFDLS
TDKDKFISGLNHLAALEYEDSDIKDVHSKESGGLNFLILFGSLASMPFE
DIAHVYEQAVANAPEASKSQVRKVFLDLLSAVGNNPHAAFGLQLVKEDK
LTDEEAEHFLAKLALNLKENSPALLTELAEVCEHVKPKRPVWVN.

The representative DNA sequence encoding SEQ ID NO: 1 as shown in FIG. 3 is:

(SEQ ID NO: 2)
ACCTCTCAGGTCAAGTACAGACTCGACGGAACTCCAGAACACTACGTCA
TCAACCACGCCTGCGCCACCTCAGAGAATGTCTTCAGGCCCTTCGGTCA
AGGGAAAACCTTCGTTGCCCAGCTCAACCGTACCTTGGATTTGGAGGAG
GTGCACGACGCCAACACCGACACTCAGCTTCCGGAGGACCTGGAGAAGG
TGCACCACATCGCGCAGACATTCCCCGAGTCTGATGAGGTGGAAAGCCT
GGAGGAACTGAAGCACGTCAACCGCTACGTGACCACCTTTGATCTCTCG
ACCGACAAAGACAAGTTCATCTCTGGTCTCAACCACCTTGCTGCATTGG
AGTACGAAGATTCCGACATCAAGGACGTTCACAGCAAGGAAAGCGGTGG
ACTAAACTTCCTGATTTTGTTCGGCTCGCTTGCCTCCATGCCCTTTGAA
GATATCGCCCACGTGTACGAGCAAGCCGTTGCCAATGCACCGGAAGCAA
GCAAGAGTCAAGTCAGAAAGGTGTTCCTGGACCTGCTCTCAGCTGTCGG
AAACAACCCTCACGCAGCGTTCGGTCTGCAACTGGTCAAGGAAGACAAA
CTCACTGACGAAGAGGCTGAGCACTTCCTCGCCAAGCTGGCGCTGAACC
TTAAGGAGAACAGCCCCGCTCTTCTGACCGAACTTGCCGAGGTGTGTGA
GCACGTGAAGCCTAAGCGACCAGTATGGGTCAAC.

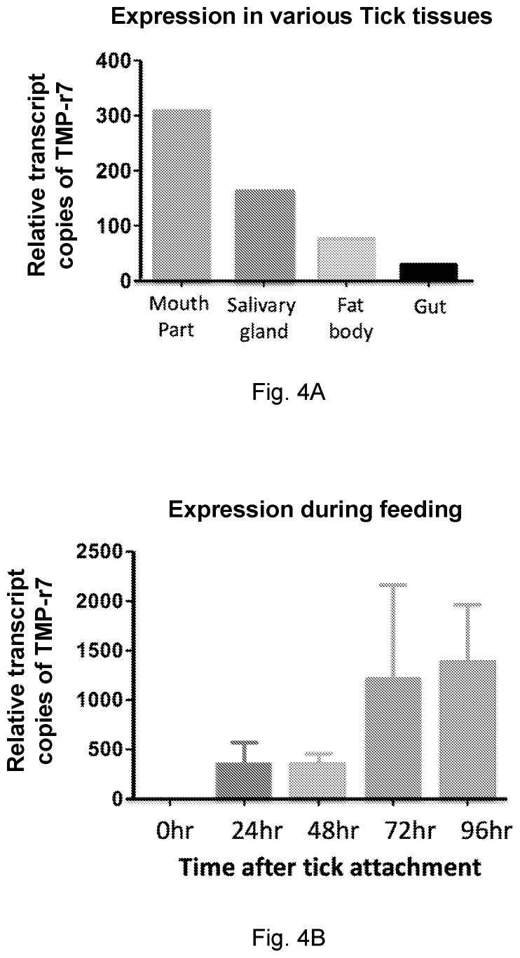

As we identified all the described TMP proteins by mass spectrometry, we next used reverse-transcription PCR analysis to assess gene expression, including one encoding the protein that includes the segment of TMP-r1 in ticks. The RT and real-time PCR analyses confirmed that TMP genes were most predominantly detected in the tick mouthparts, and to a lesser extent in various other tick tissues, as well as during the entire period of blood meal engorgement. To perform these experiments, nymphal Ixodes ticks were collected from mice at various time points during blood meal engorgement (24-96 hours) and dissected to isolate various tissues. TMP expression was assessed using primers specific to TMP-r1 via real-time PCR analysis, and normalized against tick actin gene copies. We found that the highest levels of TMP-r1-specific transcripts were detected at the tick mouthparts (FIG. 4A), but also discovered that the gene is expressed at various tick tissues and feeding timepoints, respectively (FIG. 4B). Using antisera generated against TMP-r1, we also detected production of native TMP protein in ticks. Overall, the ubiquitous distribution of TMP antigens supports their use as anti-tick vaccines.

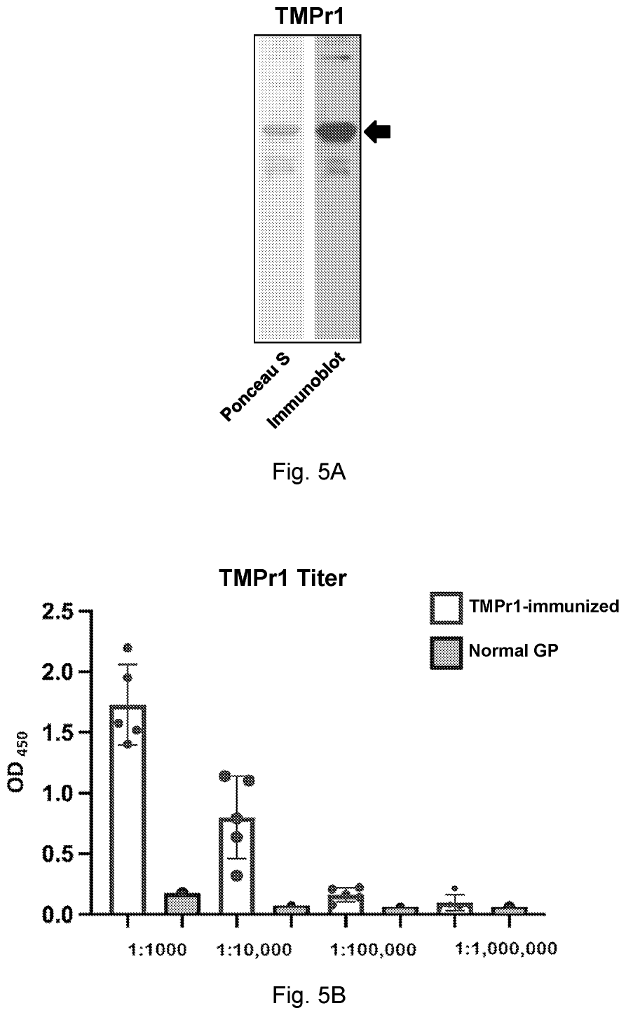

We produced these three regions as recombinant proteins in a bacterial expression system and immunized mice with 10 μg of protein in Freund's adjuvant (one primary and two booster immunizations). Immunoblot and ELISA assays indicated that the mice developed high-titer antibodies against all region-specific proteins. A separate group of mice were immunized with TMP-r1, TMP-r2 and TMP-r3 and were allowed to be engorged by nymphal ticks. The results showed that ticks were less successful in attachment and feeding on the mice immunized with TMP proteins (FIGS. 5A and 5B). While up to 83% of ticks successfully attached and engorged on the control (PBS-immunized) mice, those that parasitized TMP-immunized mice were impaired in their ability to attach (as low as 38% were recovered as fed ticks). However, as mice are the natural host of ticks and initial vaccination of TMP proteins produced variable protective immunity results, all subsequent studies were conducted using other model animals, such as guinea pigs, which are immunologically more similar to humans and represent a better model to study protective immunity against tick-bite as well as the assessment of anti-tick vaccines. Because we determined that TMP-r1 had the most dramatic effect in comparison to the other TMP proteins (TMP-r2 and TMP-r3), the additional data described herein were conducted using the TMP-r1 protein as the most effective vaccine candidate.

### Example 2

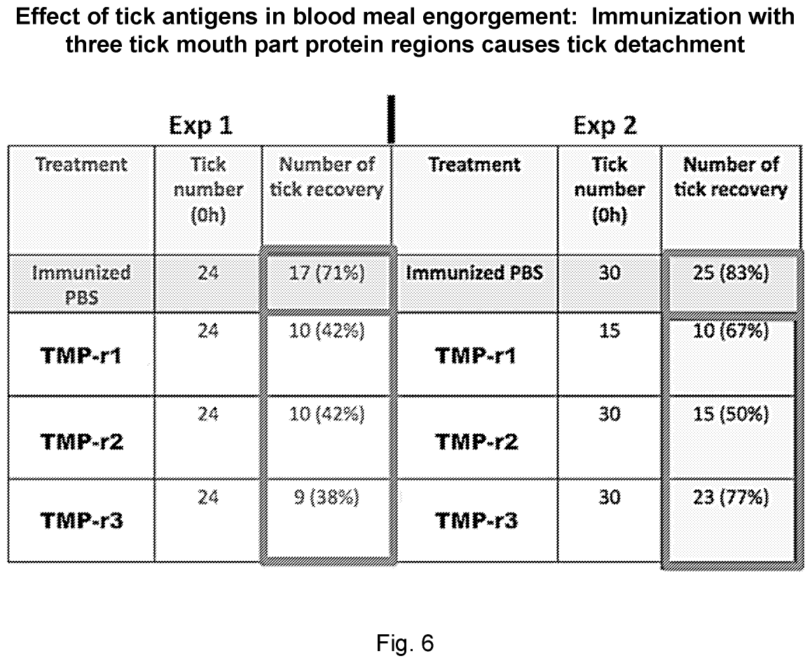

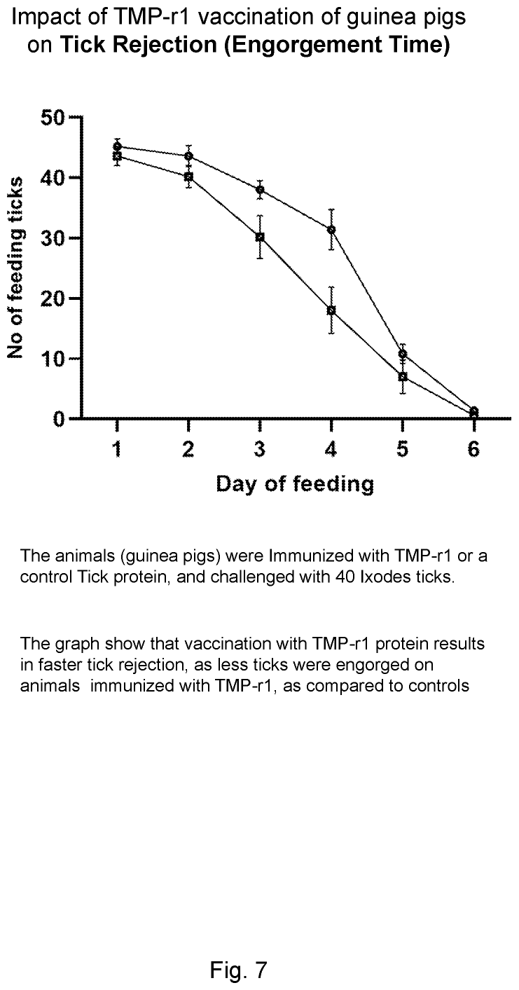

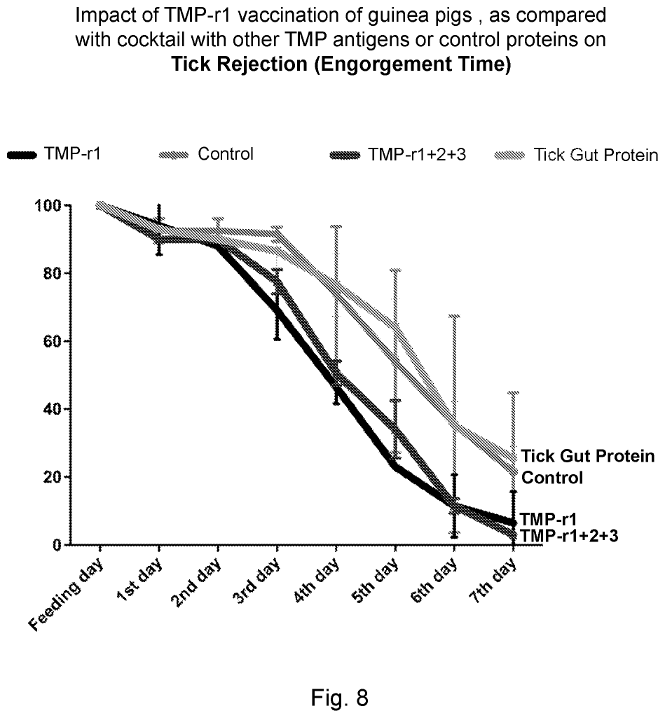

When groups of guinea pigs were immunized with TMP-r1 and a control protein, and later challenged with naïve ticks, the animals vaccinated with TMP-r1 were able to reject ticks more successfully than the control animals (FIG. 6). Further immunization studies showed that TMP-r1 alone is sufficient to elicit the anti-tick vaccine effect, as immunization with a cocktail of all TMP antigens (TMP-r1, TMP-r2, and TMP-r3) demonstrated a similar effect as TMP-r1 alone, while the controls (including adjuvant only or other tick gut proteins) failed to influence the tick engorgement process (FIG. 7). We also determined that the engorged ticks weighed less after feeding on TMP-1 vaccinated hosts, as compared to the controls (FIG. 8). In addition to Freund's incomplete adjuvant, TMP-1 immunization is also effective when using safer adjuvants like alum, and thus the disclosure includes use of the described protein with other adjuvants that may further enhance the vaccine efficacy of TMP-r1. Finally, as TMP-r1 immunization impairs successful tick feeding, and because vaccinated animals reject ticks much faster than control (non-TMP-r1) animals, we explored whether TMP-r1 vaccination could impair tick-transmitted B. burgdorferi infection.

### Example 3

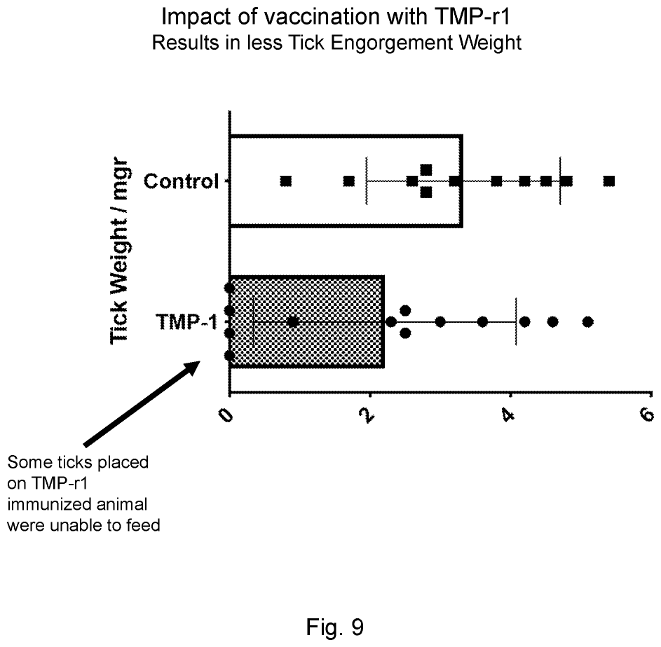

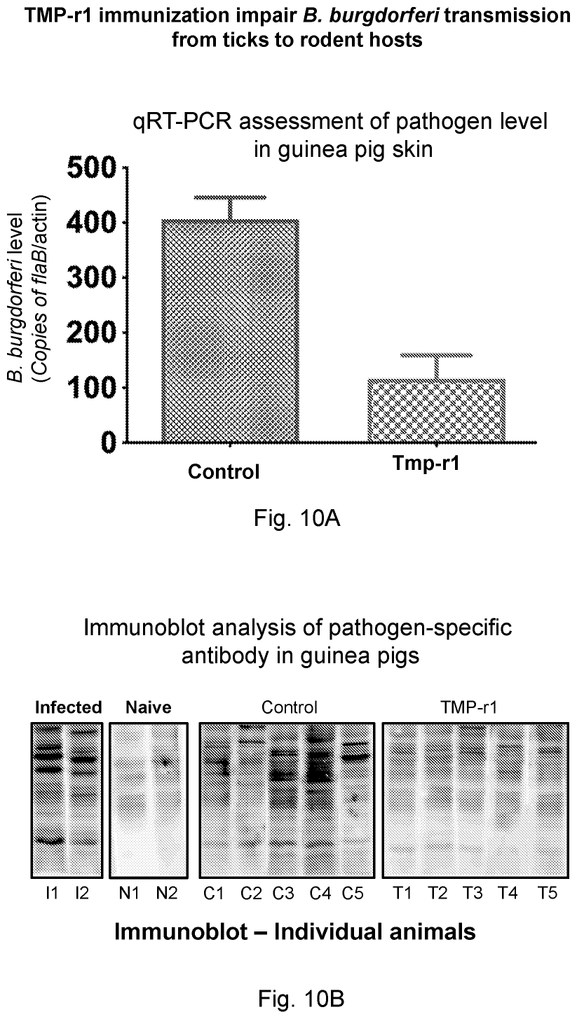

Groups of guinea pigs were immunized with TMP-r1 or a control protein, such as TMP-r2. Once these animals had generated antibodies (after three immunizations), they were challenged with B. burgdorferi-infected ticks (7-10 infected ticks/animal). After tick repletion, guinea pigs were assessed for B. burgdorferi infection using RT-qPCR and immunoblot analyses, as detailed in our publications 14-15. The data indicated that TMP-r1 immunization impairs infected tick's ability to acquire a full blood meal from the host, as reflected by their lower average engorgement weights (FIG. 9). These B. burgdorferi-ticks, which show impaired feeding capability in TMP-r1 immunized hosts (FIG. 9) were also less capable transmitting the infectious agents like B. burgdorferi to the host, as examined by the qRT-PCR assessment of pathogen level in guinea pig skin (FIG. 10A) and serological analysis of pathogen-specific antibody in guinea pigs (FIG. 10B). Taken together, these results demonstrate that the identified region of the tick mouthpart antigen, TMP-r1, is useful as an anti-tick vaccine, as it can impair successful tick engorgement and infection with Lyme disease pathogens. Further, we demonstrate use of TMP-r1 did not induce skin inflammation. Therefore, without intending to be constrained by any particular theory, it is considered that in immunized hosts, the mechanism of tick rejection, as induced by tick mouthpart antigens like TMP-r1, is likely to be independent of skin inflammatory episodes, and likely involves a novel mode of action.

The following reference listing is not an indication or admission that any particular reference is material to patentability.
1 Parola, P. & Raoult, D. Ticks and tickborne bacterial diseases in humans: an emerging infectious threat. Clin Infect Dis 32, 897-928, doi: 10.1086/319347 (2001).
2 Mead, P. S. Epidemiology of Lyme disease. Infect Dis Clin North Am 29, 187-210, doi: 10.1016/j.idc.2015.02.010 (2015).
2a. Adrion E R, Aucott J, Lemke K W, Weiner J P. Health care costs, utilization and patterns of care following Lyme disease. PLOS One. 2015; 10(2):e0116767.
3 Piesman, J. & Beard, C. B. Prevention of tick-borne diseases. J Environ Health 74, 30-32 (2012).
4 Piesman, J. & Eisen, L. Prevention of tick-borne diseases. Annu Rev Entomol 53, 323-343, doi: 10.1146/annurev.ento.53.103106.093429 (2008).
5 Pritt, B. S. et al. Identification of a novel pathogenic Borrelia species causing Lyme borreliosis with unusually high spirochaetaemia: a descriptive study. Lancet Infect Dis, doi: 10.1016/S1473-3099(15)00464-8 (2016).
6 Krause, P. J., Fish, D., Narasimhan, S. & Barbour, A. G. Borrelia miyamotoi infection in nature and in humans. Clinical microbiology and infection: the official publication of the European Society of Clinical Microbiology and Infectious Diseases 21, 631-639, doi:10.1016/j.cmi.2015.02.006 (2015).
7 Rodriguez-Mallon, A. Developing Anti-tick Vaccines. Methods Mol Biol 1404, 243-259, doi: 10.1007/978-1-4939-3389-1_17 (2016).
8 Sprong, H. et al. ANTIDotE: anti-tick vaccines to prevent tick-borne diseases in Europe. Parasites & vectors 7, 77, doi: 10.1186/1756-3305-7-77 (2014).
9 Valle, M. R. & Guerrero, F. D. Anti-tick vaccines in the omics era. Front Biosci (Elite Ed) 10, 122-136 (2018).
10 Willadsen, P. Anti-tick vaccines. Parasitology 129 Suppl, S367-387 (2004).
10a. Parola, P. & Raoult, D. Ticks and tickborne bacterial diseases in humans: an emerging infectious threat. Clin Infect Dis 32, 897-928, (2001), PMID: 11247714.
11 Sonenshine, D. E. Biology of Ticks. Vol. Vol-1 (Oxford University Press, 1993).
12 Ohnishi, J., Piesman, J. & de Silva, A. M. Antigenic and genetic heterogeneity of Borrelia burgdorferi populations transmitted by ticks. Proc Natl Acad Sci USA 98, 670-675. (2001).
13 Richter, D., Matuschka, F. R., Spielman, A. & Mahadevan, L. How ticks get under your skin: insertion mechanics of the feeding apparatus of Ixodes ricinus ticks. Proc Biol Sci 280, 20131758, doi:10.1098/rspb.2013.1758 (2013).
14 Kariu, T., Smith, A., Yang, X. & Pal, U. A Chitin Deacetylase-Like Protein Is a Predominant Constituent of Tick Peritrophic Membrane That Influences the Persistence of Lyme Disease Pathogens within the Vector. PLOS ONE 8, e78376, doi: 10.1371/journal.pone.0078376 (2013).
15 Yang, X. et al. Analysis of Borrelia burgdorferi Proteome and Protein-Protein Interactions. Methods Mol Biol 1690, 259-277, doi: 10. 1007/978-1-4939-7383-5_19 (2018).
16 Yang X, Coleman A S, Anguita J, Pal U. A chromosomally encoded virulence factor protects the Lyme disease pathogen against host-adaptive immunity. PLOS Pathog. 2009; 5(3):e1000326. PubMed PMID: 19266024.
17 Kumar M, Yang X, Coleman A S, Pal U. BBA52 facilitates Borrelia burgdorferi transmission from feeding ticks to murine hosts. J Infect Dis. 2010; 201(7): 1084-95. PubMed PMID: 20170377; PMCID: 2832101.
18 Yang X, Smith A A, Williams M S, Pal U. A Dityrosine Network Mediated by Dual Oxidase and Peroxidase Influences the Persistence of Lyme Disease Pathogens within the Vector. The Journal of biological chemistry. 2014. PubMed PMID: 24662290; PMCID: 4007469
19 Singh P, Verma D, Backstedt B T, Kaur S, Kumar M, Smith A A, Sharma K, Yang X, Azevedo J F, Gomes-Solecki M, Buyuktanir O, Pal U. Borrelia burgdorferi BBI39 paralogs are targets of protective immunity inducing microbicidal responses and reducing pathogen persistence either in hosts or in the vector. J Infect Dis 215(6): 1000-1009, 2017. PMID: 28453837 PMCID: PMC5407057
20 Kung F, Kaur S, Smith A A, Yang X, Wilder C N, Sharma K, Buyuktanir O, Pal U. A Borrelia burgdorferi Surface-Exposed Transmembrane Protein Lacking Detectable Immune Responses Supports Pathogen Persistence and Constitutes a Vaccine Target. J Infect Dis. 2016; 213(11): 1786-95. PubMed PMID: 26747708; PMCID: PMC4857468.

1. A method for inhibiting persistence of tick attachment to a mammalian host, and/or inhibiting transmission of one or more tick-borne pathogens from a tick to the mammalian host, the method comprising administering to the mammalian host an isolated or recombinantly produced protein that has at least 95% sequence identity with the sequence of SEQ ID NO:1.

2. The method of claim 1, wherein administration of the protein inhibits the persistence of the tick attachment to the mammalian host.

3. The method of claim 1, wherein administration of the protein inhibits transmission of the one or more tick-borne pathogens to the mammalian host, and wherein optionally the one or more tick-borne pathogens include Borrelia burgdorferi.

4. The method of claim 1, wherein administration of the protein inhibits the persistence of the tick attachment to the mammalian host and inhibits transmission of the one or more tick-borne pathogens to the mammalian host.

5. The method of claim 1, wherein the mammalian host is a human, a canine, a feline, or an equine.

6. The method of claim 5, wherein the administration comprises a subcutaneous administration.

7. The method of claim 5, wherein the isolated or recombinantly produced protein is administered as a component of a composition, the composition further comprising an immuno-effective amount of an adjuvant.

8. The method of claim 5, wherein the isolated or recombinantly produced protein is administered as a component of a composition, the composition further comprising at least one additional tick immunogenic protein or peptide.

9. A vaccine composition comprising an isolated or recombinantly produced protein that has at least 95% sequence identity with the sequence of SEQ ID NO:1.

10. The vaccine composition of claim 9, wherein the composition further comprises an immuno-effective amount of an adjuvant.

11. The vaccine composition of claim 9, further comprising at least one additional tick immunogenic protein or peptide.

12. The vaccine composition of claim 9, wherein the isolated or recombinantly produced protein consists of the sequence of SEQ ID NO:1.

13. A recombinant polynucleotide encoding a protein that has at least 95% sequence identity with the sequence of SEQ ID NO:1.

14. The recombinant polynucleotide of claim 13, wherein the polynucleotide is an RNA polynucleotide.

15. The recombinant polynucleotide of claim 14, wherein the RNA polynucleotide is an mRNA polynucleotide.

16. The recombinant polynucleotide of claim 13, wherein the polynucleotide is a DNA polynucleotide.

17. The recombinant polynucleotide of claim 14, wherein the polynucleotide is present in an expression vector.

18. A prokaryotic cell comprising the expression vector of claim 17.

19. A method of making a protein for use as a tick vaccine, the method comprising expressing the protein from an expression vector in a plurality of prokaryotic cells and separating the expressed protein from the cells.

20. The method of claim 19, further comprising purifying the protein that is separated from the cells.

21. An isolated or recombinantly produced protein that has at least 95% sequence identity with the sequence of SEQ ID NO:1.

22. The isolated or recombinantly produced protein of claim 21, wherein the protein consists of SEQ ID NO:1.


SEQUENCE LISTING (8 sequences)

SEQ ID NO: 1 - protein, 240 aa, Ixodes scapularis
TSQVKYRLDGTPEHYVINHACATSENVFRPFGQGKTFVAQLNRTLDLEEVHDANTDTQLPEDLEKVHHIAQTFPESDEVESLEELKHVNRYVTTFDLSTDKDKFISGLNHLAALEYEDSDIKDVHSKESGGLNFLILFGSLASMPFEDIAHVYEQAVANAPEASKSQVRKVFLDLLSAVGNNPHAAFGLQLVKEDKLTDEEAEHFLAKLALNLKENSPALLTELAEVCEHVKPKRPVWVN

SEQ ID NO: 2 - DNA, 720 bp, Ixodes scapularis
acctctcaggtcaagtacagactcgacggaactccagaacactacgtcatcaaccacgcctgcgccacctcagagaatgtcttcaggcccttcggtcaagggaaaaccttcgttgcccagctcaaccgtaccttggatttggaggaggtgcacgacgccaacaccgacactcagcttccggaggacctggagaaggtgcaccacatcgcgcagacattccccgagtctgatgaggtggaaagcctggaggaactgaagcacgtcaaccgctacgtgaccacctttgatctctcgaccgacaaagacaagttcatctctggtctcaaccaccttgctgcattggagtacgaagattccgacatcaaggacgttcacagcaaggaaagcggtggactaaacttcctgattttgttcggctcgcttgcctccatgccctttgaagatatcgcccacgtgtacgagcaagccgttgccaatgcaccggaagcaagcaagagtcaagtcagaaaggtgttcctggacctgctctcagctgtcggaaacaaccctcacgcagcgttcggtctgcaactggtcaaggaagacaaactcactgacgaagaggctgagcacttcctcgccaagctggcgctgaaccttaaggagaacagccccgctcttctgaccgaacttgccgaggtgtgtgagcacgtgaagcctaagcgaccagtatgggtcaac

SEQ ID NO: 3 - protein, 1329 aa, Ixodes scapularis
MRVPYVLPVLFLAAASAFEVGKDYVYHYNGKMQVYNPEQPLQSSGFAFRSKVVAQPRPDHTHFKIIDFEVDSFNGDHVHVGEHEFNYHSTEALKQFIERPFAGKFSEGKLEEAELSKSEPKWARNLKKGVLSIFQLDLVKGRHDHPHAKQFHVREEGLHGNCDTLYVVAEEEGHLKVTKIKNLEKCDKEHYAVYGRIKGHECVDCEAQETHPFVATSQVKYRLDGTPEHYVINHACATSENVFRPFGQGKTFVAQLNRTLDLEEVHDANTDTQLPEDLEKVHHIAQTFPESDEVESLEELKHVNRYVTTFDLSTDKDKFISGLNHLAALEYEDSDIKDVHSKESGGLNFLILFGSLASMPFEDIAHVYEQAVANAPEASKSQVRKVFLDLLSAVGNNPHAAFGLQLVKEDKLTDEEAEHFLAKLALNLKENSPALLTELAEVCEHVKPKRPVWVNCQLALSTLAGQEGCVRAKTDKEQDEGFCKPSIVSHFFNYEIKPEDKKDQPEYKRTVYMKAAGNLATRGAVHYLERYVSDTNQPEYRRSAALWAMVRAAPHHHELVRDVALPLYKNKSETAYLRIGAFVNVLMTKPDLYLLKYIGHNIIDDPSDQLASYVTSAFRSLVKSKYPCHQELAQHLRYVVPMWNDVSRFAKPLDHTKSQVILSSGYDPKYDYGGSSVFAQVRADDSYLPREVYFSAKDYFHGRSFESVSLKFESWGLDKLLNHVVGPQPGSTKNLWNVFGRRRFTRDASAKDLQEVEAALPITDRDYDHVYGRLSLDLYGHAFDTWEFDESILQELSKGEGEEAPGDKLKNLLGESKRKKSFILSKDICVMVPSELGVPVFYHILQVEYMYINPKKFAFDQTQDAKFAVDLERHYVHESRGYSMFGVALTFNKTSMGTGSDSQTIMNLPIDLKITLDPIHKKLSMKRPLSLPWNVLNHHFRPFTFVMSYDMHKDATTTETMMTQAMHPLYNKEELTEFDRNYFGEVLGYGLNVKGSLLSKGLKKGLHDYWYESDDRQKFYYATLNPEWHPRELQFTVVPAAQDATTEVEVDLGYKFLEPDDPRESHFEAHDNVGDDAEVPSTHVLNLDYTLKGTKERKVSAELRYSFTKDLLKHKVQLFYDRTPFHPKEQDHTKVEVDLGYKFLEPDDPRESHFEAHDNVGDDAEVPSTHVLNLDYALKGTKERKVSAELRYSFTKDLLKHKVQLFYDRTPFHPKEQDHTKITFKGKYTHTDDEEKQIRENAEGKPLGINRMKKYTLRRPYKKCTEHQKRGVPLNFECMKYLYYTSRLGKLTTDVEYKNLKPVSVEGLHPVNQGRAPASFRRFRTGAPR

SEQ ID NO: 4 - protein, 1555 aa, Ixodes scapularis
MRVPYVLPVLFLAAASAFEVGKDYVYHYNGKLQVYNPEQPLQSSGFAFQSKVVAQPRPDHTHFKIIDFEVDTFNGEHVHLSDHQFHYHSTDALKQFIERPFAGKFSEGKLEAAELGKSEPMWSQNIKKGVLSVFQLDLVKGRHDDPHAKQFYVKEDGLHGNCDTLYVVAEKEGHLEVTKIKNLEKCDKDHFAIYGRIKAHECVDCEAQESHPLVATAQVKYRLDGTPEHYVIDHACATSETVLRPYGKGKTFVVQLNRTLDLEEVHDANTDTQLPEDLERVDHLSQTLPVGDHVETLQDLKKVNHFVDYFQLTNNKEKFVAGLNHLAALEYEDDDVKDVHGKESGGLQFLVLFNALSTLHFEDVVQVYEQAVASAPEASQSHVKRLFLDLLSAAGTNPQVAFGLQLVKEDKLSDDEAEHFFTKLALNLKENSPALLTELAEVCEHVKPKRQVWVNCQLALSILAGQEGCVRAKTDKEHDEGFCKPSLVSHFFNYSVKPEDKKDQPEYKRTAYIKAAGNLATRGAVHYLERFISDTNQPEYTRAAAFWALRRAGSKHPELVRDIALPVYKNKSESSYLRIAAFTAVMKTHPDLYLLKYIGHNIIDDPSDQVASYVTSLFRSFEKSKYPCHQSLAQHLRYVVPMWNDVYRFSKPPDYTKSHVHLSSGYDPKYDYGGATSFGMVRADDSYLPRDVYVSVKDYMSGHSLSTATLWFENWGMDKLLNHVVGPQPGSSKNLWNVFGRRRFTRDASAKELKEVEDALPVTDREYDHVYGRLNLDLFGHAIDSWEFDEDLLKEVGKEDGEGKCLESLKLLILTDRLRKHKPGTKLKNLLGQVQRKKNFYLSTDIIFVAPTEVGVPIFFDFKQAEFTYINRKKFSVDQTEDAKFTFDVQRHYVHESRGYTMVGVGLTFNKTSLGTGSDVQTIFNLPIDLHLTVDPIHKTLSLKRPLSLPWNVLNHHFRPFTFTMPYDIATDVSHAVTTLEQPQYPLYNKEEVTEIDHTYLTDVFGYGLNVKGSLLSKGLKKGLHDFWYESDCRQRYYYALMNPEWHPRELQFTVVPAEHDATTEVEVDLGYKFLEPDDARESHFPVQDNVGGDAEVPSTHVLSLDYTLKGSKERKASAELRYSFTKDNLKHKVQFFYDRTPFHPKEQVHTKVCMDVTSKFPKPEWSKLKNLAVFHEGKDLESELNLHYGSNCVDQSSITFKGKYTHTDDEERQIRENAEGKPRGINRMDKYNLRDPFNKCTEHQKRGVPLNLYCMKYLYYTSRLGKFTTDVEYKNLKPLLPMLTKYYKKVHPEGGFLSTVVSHFHGPNGKLHVVSQVPPVQDYADIVVTTEDGHSHNHSHVPLYSHLLEPRIMFSTLGYSNMAHYISYYKQKYCDLQGQSVRTFDNVVVQLPETDCYKVVAKDCSPNKRFTVLARATGNAELPKALKAFIQTTQVELLPVAEDSGLVLRVDGNRVPLTPGVPYSHTAHDAELFTVTLENKYFEVTSKPYGLNLGFNGNMLFVQTANFYRGKLCGLCGDYNYDRQHELVGPNLHHYNDTLEFAKSYVVPSSDCTP

SEQ ID NO: 5 - protein, 1494 aa, Ixodes scapularis
AGYEVGNVYVYHYNGKLQVQNPEQPLQSSGFVFRSKVIAQPSPDGTHFKIIDFEVDSFNDEHTQLSEQEFHYHSTDALKQFIERPFAGKFSKGKLVAAELGKNEPKWSKNLKKGVVSVFQLDLVNGRHDNPNAKQFYVKEESPLGNCDTLYIVDENEGDLRVTKIKNLKKCDEQIYTIGRVKGHKCVDCKALETHPGLATSQIQYSLDGTPEHYVINHASATSDTEFKNFGAGNTIRVQINRTLELEEVRAATTDVRLPEDIEKVDHLWLSFPESGGADSLEELKEVNRFVADYEFTAVKDRFIAGLNYLVSIEYGDGDIRDVDSKEIGNNFLALHSSFAAMPFEDVAQMYDQTVANAPEASKSDVRRLFLDLLSATGGNPHAAFGMQLIKEDKLSDDESEHFLSKLALHLKENSPALLTELASICEHVKPRRHVWVNCQLALSSLAGQKGCVRAKTNKEHDKGICKPSLVSHFFNYSITPSDKKDQPEYKRTVYIKTAGNLATRSAVLYLERFISPEANQPEYRRAAAFWALKQSVPHHPEMVRDIALPVYMNKSESSYLRITAFVNVLLTNPDLYLLKYIGHNIIDDPSDQVASYVTSRFRALVNSKYPCHQELAHHLAHVVPMWNNVDRFSEPLDHTQSQFTLSTSYDPKYDYGSATAFGLVRADDSYLPRGAYLGVQDYFGGHSSNTVSLTFENWGLDKLLNRVLGPHPGSTTNMWNVFGRRRFTRDASVEEIKEVEDALPVTDREYDHVYGRLSLDLFGHAIDTWEFDESIVQELTEGDDTPGRKLRNLLGKVQRKRSFSLSKDLLYGIPTELGVPIYIEHKQAECTYINRKTFSVNQADDAKFSLDFKRHYVHQSQGYSVVGVNLAFNKTFLAVGANVQTLFNLPINLHVTVDRVHKRMSLKRPLSLPWNVMNHHFRPYTFVKQYGLATDASTTAPYLSQYPVYNNHEVTEFDRTYFADIFGYGLNVKGSLLSKGLKKGLQNFWHESDYRQKYYYATQNPEGHPRDLQFTLVPAEGDATTEIQVDLGYNFLEPNSPRDSHFPVHDNVGEDGQVPSTHVFNLDYALKGTKERKVSAELRYSFTKDNFRHKVQFFYDRTPFHPEEKDHTKVCLDVTAMFPEPDWARLNNLSLFYEGKKLESQLYLRYGKNCVDHSSIIFKSKNTHTDDEAKQIRENAEGQPLGNNRLKMYKLRWLFKKCTEDQRRGVPLNFCMKYMYHSSRLGKFATEVEYKNLKPLFSTLLVHHMTTDPHGSFLGTLASHIHGPNGKLHVVSQVPPVEEYSDIVVTTEDGHSFHHEHVPIYSHLLAPRVFSLLGYSNMADYISYYTQSKFVGVFFFGVTTFDDVVVNLPETDCFKVVAKDCTPHKRFAVLARATGVTKHPKALKVFIQSTKVELFPASHGAGLEIYIDGNRVPLTPGVPYSHTAHDVELFTVTQADKFYEVTSLPYGLFLGFDGNILLVQTANIYRGKLCGLCGDYNYERQHELVGPDP

SEQ ID NO: 6 - protein, 1325 aa, Ixodes scapularis
HPGLATSQIKYRLDGTPEHYVINHADATSDTEFKPFGTGGKTIRVQINRTLDLEEVHDANTDTQLPEDIEKVEHLWLSFPESGDAESLEDLKKVNHPVIDFDLTNDKEKFIPAFNQLAATEYEDDDIKDVHSKESVALKFLILHSLFGVMPFEDVSQMYDQAVANAPEAGKSNVRRLFLDLLSATGTNPHVAFGLQLIKEDKLSDDEAEHFLSKLALHLKENSPALMTELSEACEHVKPKRQVWVNCQLALSSLAGQEGCVRAKTDSEHDEGLCKPSLVSHFFNYQIKPEDKKDQPEYKRTVYMKAAGNLATRGAVHYLERYVSDTNQPEYRRSAALWALKRSSNHHPELTGKQPVFKNTHKEKLGKIFLFMATESNYSYLDHDVHYNDAKEANSKIDSNTLFLNLCAFPQKKKKSKNRHRNPQHLRYVVPMWNDVPRFDKPLDHTKSQLLISSGYDPKYDYGGATTFGQIRADDSYLPREVYLGFKHYYSGASYETASLWFESWGMDKLLNHAIGPRPGSTANMWNVFGRRRFPRDASAKELKEIEDALPVTDREYDHVYGRLSLDLFGRAIDTFEFDESLFEEAANARGEFLDSFRHEVPRGKQLYQSASKLESFLEPESPAPISTQEVKPYYEVYLLNQVGVCHDDSWLRHVAYGIAKCSKNPSRTSNMVGVGLTFNKTFVGVGADIQTVFNLPIDLKVTVDPIHKKLSLNRPLSLPWNIVNHHFRPYTFAMPYDLVSDVSTAVTALSQPQYPLYNKDEVTSFDRTYFTDLLGYGLNVKGSLLSKGLKKGLHDFLLHSDCSKMLYYAVWNPEWHPRELQLTVVPDEQDATTEVEVKLGYEFLEPDDPRESHFPVNDNVHGDAEVPSTHVLNLDYALKGTKERKVSAELRYSYTKDNLKHKVQFFSDRTPFHSMDQDHSKICLDVTTKFPEPDWDRLNNLAVFHEGKELEFQMNIHQGSNCIDQASVSFTGKYTHTDDEAKQIRENAEGKPLGANRMKKYNLRVPYNKCVEHQKQGVPLNYYCIKYLYYTSRLGKLTMDIVYKNRWDLFSPMPHYYKKINREGGFVSTLSEYYRRATSGKLHVVSQVPPVEKYADIAVTTEDGRIYNHFHVPIYSHLLEPKIFPMLGYSNMANYISYYKHTNTAIISCLFVPFYTPGNLVLNWHRSPQPRRLQNLKFTGTFYRHSPSVILLQALKAFIQSTQVELLPVAEDSGLVLRVDGNRVPLTPGVPYSHTAHEVELFTVKMEHNYFEVTSKPYGLYLGFNGNMLVVQTANFYRGKLCGLCGDYNYDRQHELVGPNLHHYNETLEFAKSYVVPSSDCTPP

SEQ ID NO: 7 - protein, 1111 aa, Ixodes scapularis
MSLQNRALEDSAFETGKEYVYHYNGRLQIKNPEQPLQSSGFAFRSKVVAQPKSDHTYFKITDFEVDSFNGDRIILDDHQFNYHSTDDLREFIERPFAGTFSHGKIVEIAIGREEPLWSKNLKKGVLSTFQLDLVTGRQNNPHVEEYTTREHGLHGNCETKYLVTEKEGHIEVFKVRDLDLCDKEAHIVYGRIKGHKCVECEAMETHPLSSTFEVKYRLDGTPQHYVIKHASAASDTLFQPFGAGKAVRVVVNRTLFLDEERAANADMQLPDDVGVSDELTHSFSEYGKAQTLEDLKHPNHYITDSKLQNGRQKFVEGLNHLAAFGYDDSDIGSVDTKESGALRFLLLFRSMASMRYEDIEQVYEQNVVNAPEPNRQNVRRLFLDVLAAAGSNPHVAFGLKLLKEGKLSGGDAEHFLTRLPLNLKEHSPALITQLAVRLRLFRSVQHLRYVVPLWDNVQRFSKPLDYTQSQLALSSGYDPEYDYGGATIFSMIQADDSYLPRSLYLKAKDYYAGYSYDTIAMSFEGWGLNKLIDGVAGPRPGSTKNLWNFGGRRRFTREASAKDRKEIEDALPIQDWPFDPFYARIGLSMYGNEIDTWEIDERMLAELKESDKPGDDPWDILTKKVRRKSFFLNNDYVLLVPTDMGVSVFMDFKSVDFAFIDRQKFDFARTADDKFSLDVKRRYVFENRGFSVVGVALTFNETAVGSGSDSRTVLSLPLELKATLDTLNHKLHLQRPLALPWNILNHHNRPSTFALPFDPTRDIGNAVIQLADPPTPLYNKFELTEIDRGFRTDSLPIGLNVEGSMLSKGLSEGLHKFWYEMDWRQKFYYAAMNPHWHPRELRLTLVEAEKNFPTTIELDLSHKFLKPEDARESKFPITFNGKFTHTDDDAQQIRENAEAKPRSPNRLRPYYLRDMYAKCVHHQKLGIPLNYYCVKYMYYSSRLGKLTMDVEYKKLKPLLPGLYYHMTEYHSHSEDHVGFFGVIGEHMDGPTGKLRVVSQVPPRHKCADVLVSTSDGHHFFHPHVPLYSHILEPRVFVAFGYSNIAEYTAYYKQSEYGPPTASFYRGSLCGLCADYNYDRLNELLGSDHQVHNDTLEFAESYVVPSSDCTPP

SEQ ID NO: 8 - protein, 480 aa, Ixodes scapularis
MIYQTCVVKYHNTWNDAKENAICTPTMAAKYFNYLVPQYERNLEEKERTMYIKVAGNFGLKEALPFLEKYAEDKMDQFPVHIRMISIWVLEKAALYNPEKVRDIVLPIYRNWTESHEIRLAAFTILMRTSPDMALLKSIAAELVTTEPNTQVISYVWSTFKHMAELDYPCYFEIARHLEYVMPIIEGHERLKDLEEWPYSKKIINGGYEPEFDYGGYSVFSYIMANDSYMPRSMYIKMNDYMNNFNYDTFSASFDGWGLEPILDKMFGPKVKGDISPGRSLWNFFGRRRSTRDIAVHKEVKEIDQNLHIETRNHPKPYAALRVTAFNNELINLNFDEEMFDTIMTSGENRPMSILRKIVGDIKDFQVRSFMMDGDMSMFLPTEIGLPFYLERKQPMFMYYKNKEMKFDVKGNEADGTIDEITTKINGHFMFDHHTIETASILVPFEKISVGVGYDSRIAMSIPVDIDAGMNMVDRKFRMK